(19) United States
(12) Patent Application Publication
(10) Pub. No.: US 2011/0166541 A1
(43) Pub. Date: Jul. 7, 2011

(54) ADJUSTABLE SOLID PARTICLE APPLICATION SYSTEM

(75) Inventors: Oskar Stephan, Hockenheim (DE); Reiner Witt, St. Leon-Rot (DE); Ulrich Schröder, Frankenthal (DE); John Joseph Louden, Manchester (GB); Xiaomin Zhang, Charlotte, NC (US); Martin Juarez-Zamacona, Tega Cay, SC (US)

(73) Assignee: BASE SE a German Corporation, Ludwigshafen (DE)

(21) Appl. No.: 13/063,075

(22) PCT Filed: Sep. 14, 2009
(86) PCT No.: PCT/EP2009/061859

§ 371 Date: Mar. 9, 2011

Related U.S. Application Data
(60) Provisional application No. 61/097,458, filed on Sep. 16, 2008.

(51) Int. Cl. A61L 15/60; B05B 1/00; B05B 15/00; B05D 1/12; B05D 5/04

(52) U.S. Cl. 604/372; 118/308; 427/180

(57) ABSTRACT

An application system for applying a solid particle includes an injector housing having a material inlet, a gas inlet, and a material outlet, and an occluder moveably disposed within the injector housing between the gas inlet and the material outlet. The injector housing and the occluder define there between at least one adjustable aperture downstream of the material inlet and in fluid communication with the gas inlet and the material outlet. The aperture has a first open area with the occluder in a first position and a second open area with the occluder in a second position, the second open area being different than the first open area. The system also includes a nozzle having a nozzle inlet coupled to the material outlet and a nozzle outlet, the inlet having a circular cross-section and the outlet having a rectangular cross-section.

This disclosure is directed to a system for applying solid particle material to a substrate to manufacture, for example, absorbent articles, such as baby diapers, adult incontinent products, feminine hygiene products and the like. In particular, this disclosure relates to a system that permits the solid particle material, including superabsorbent polymer (SAP) particles, to be adjustably applied to a target region of the substrate in a uniform pattern.

In a typical air-laying process, SAP particles are applied to a substrate to form an absorbent core for absorbent articles, such as baby diapers, adult incontinent products, and feminine hygiene products. Conventional SAP application systems lack the ability to apply the SAP particles uniformly (i.e., in a controlled manner) to the substrate. Moreover, conventional SAP application system lack the ability to control the amount of SAP applied, other than by varying the output of the associated source of pressurized air.

FIG. 1 illustrates a conventional applicator 10 for use with SAP particles 12. The applicator 10 is defined by a conduit 14 with a cylindrical cross-section. The conduit 14 has a wall 16 that encompasses a flow region 18. When the SAP particles 12 are pneumatically transported through the applicator 10, a non-uniform airflow 20 typically develops within the flow region 18. As illustrated in FIG. 1, the non-uniform airflow 20 has a substantially helical shape, although other non-uniformities (whether spatially dependent, time-dependent, or both) may be encountered.

Because of the flowability difference between the SAP particles 12 and the conveying air, centrifugal forces induced by the non-uniform airflow 20 tend to segregate the SAP particles 12 within the flow region 18. When the SAP particles 12 reach the end of the applicator 10, they tend to be non-uniformly distributed across the cross-section of the conduit 14, as seen in FIG. 2. Moreover, FIG. 2 illustrates the time-dependent nature of the SAP particle distribution 22 resulting from the illustrated helical non-uniform airflow 20 as the particles exit the applicator 10.

The non-uniform, time-dependent nature of the SAP particle distribution 22 has a pronounced effect on the formation of a particle-substrate composite, such as is used in the manufacture of absorbent articles, such as baby diapers, adult incontinent products, and feminine hygiene products. The effect of the non-uniform airflow 20 on a particle-substrate composite 24 is illustrated in FIG. 3. Ultimately, when the SAP particles are applied to a substrate 26 located on a forming surface in a forming chamber, the applied particle layer 28 is non-uniform. For example, if the applicator 10 is used to apply the SAP particles to a substrate 26 when the substrate 26 is moving relative to the applicator 10 in the y-direction, the non-uniform distribution illustrated in FIG. 3 results in the applied particle layer 28 having a local maximum thickness 30 (i.e., in the z-direction) that varies in both directions coplanar with the substrate 26 (i.e., in the x-and y-directions or, equivalently, in the cross- and machine-directions).

The non-uniform distribution of the applied SAP particles on the substrate is undesirable. Products so formed have a correspondingly variable composition, and the fraction of products that are rejected for being outside of quality control specifications increases. The weight distribution deviation in such products can be as high as 40% relative to the desired mean distribution. The inability to control the application of the SAP particles also results in other process inefficiencies, such as a loss of SAP material around the forming machine, an increased amount of SAP that must be recycled through the various screens of the forming machine, thereby degrading the process performance properties and reducing the lifespan of the various filtering media in the forming machine.

As set forth in more detail below, the present disclosure sets forth an improved assembly embodying advantageous alternatives to the conventional devices and methods discussed above.

### SUMMARY

According to an aspect of the present disclosure, an application system for applying a solid particle is provided. The system includes an injector housing having a material inlet, a gas inlet, and a material outlet downstream of the material inlet, and an occluder moveably disposed within the injector housing between the gas inlet and the material outlet. The injector housing and the occluder define therebetween at least one adjustable aperture downstream of the material inlet and in fluid communication with the gas inlet and the material outlet. The aperture has a first open area with the occluder in a first position relative to the injector housing and a second open area with the occluder in a second position relative to the injector housing, the second open area being different than the first open area. The system also includes a nozzle having a nozzle inlet coupled to the material outlet of the injector housing and a nozzle outlet, the inlet having a circular cross-section and the outlet having a rectangular cross-section.

According to another aspect of the present disclosure, a process for applying a solid particle material to a substrate using an application system is also provided. The application system includes an injector housing having a material inlet, a gas inlet, and a material outlet downstream of the material inlet, and an occluder moveably disposed within the injector housing between the gas inlet and the material outlet. The injector housing and the occluder define therebetween at least one adjustable aperture downstream of the material inlet and in fluid communication with the gas inlet and the material outlet. The application system also includes a nozzle having a nozzle inlet coupled to the material outlet of the injector housing and a nozzle outlet, the inlet having a circular cross-section and the outlet having a rectangular cross-section. The process includes disposing the occluder in a first position relative to the injector housing so that the aperture has a first open area, passing air through the aperture, drawing the solid particle material into the injector housing at a first rate, and ejecting the solid particle material from the nozzle outlet onto a substrate. The process further includes moving the occluder to a second position relative to the injector housing so that the aperture has a second open area, the second open area being different than the first open area, passing air through the aperture, drawing the solid particle material into the injector housing at a second rate, the second rate being different than the first rate, and ejecting the solid particle material from the nozzle outlet onto a substrate.

### BRIEF DESCRIPTION OF THE DRAWINGS

It is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 is a cross-sectional view of an applicator according to the prior art;

FIG. 2 is a series of end views of the applicator according to FIG. 1, illustrating the solid particle distributions within the applicator;

FIG. 3 is a perspective view of a particle-substrate composite material produced using the applicator of FIG. 1;

FIG. 4 is a side view of an application system according to the present disclosure;

FIG. 5 is a plan view of the application system of FIG. 4;

FIG. 6 is a cross-sectional view of a injector housing and an occluder of the application system of FIG. 4 taken at line 6-6 in FIG. 5, with the occluder in a first position;

FIG. 7 is a cross-sectional view of the injector housing and the occluder of the application system of FIG. 4 taken at line 6-6 in FIG. 5, with the occluder in a second position;

FIG. 8 is a perspective view of a nozzle of the application system of FIG. 4;

FIG. 9 is a cross-sectional view of the nozzle of FIG. 8 taken at line 9-9 in FIG. 8;

FIG. 10 is an end view of the nozzle of FIG. 8;

FIG. 11 is a schematic view of the application system of FIG. 4 as assembled with a production system to apply SAP to a substrate in the manufacture of an absorbent article, for example;

FIG. 12 is a perspective view of a particle-substrate composite material produced using the application system of FIG. 4;

FIG. 13 is a graph of the correlation between a diameter within the injector and the flow rate through the applicator system; and

FIG. 14 is an end view of a variant of the application system of FIG. 4.

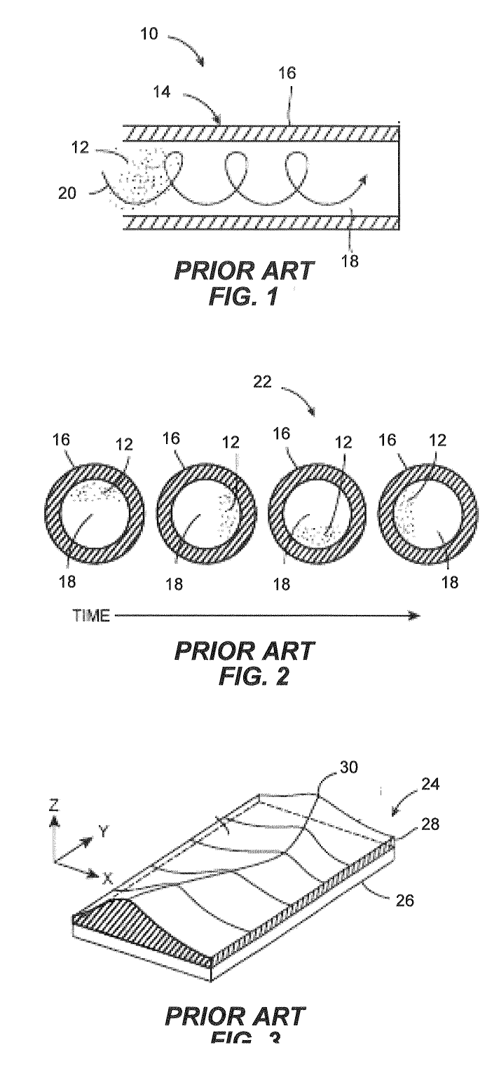

### DETAILED DESCRIPTION OF VARIOUS EMBODIMENTS

Although the following text sets forth a detailed description of different embodiments of the invention, it should be understood that the legal scope of the invention is defined by the words of the claims set forth at the end of this patent. The detailed description is to be construed as exemplary only and does not describe every possible embodiment of the invention since describing every possible embodiment would be impractical, if not impossible. Numerous alternative embodiments could be implemented, using either current technology or technology developed after the filing date of this patent, which would still fall within the scope of the claims defining the invention.

It should also be understood that, unless a term is expressly defined in this patent using the sentence “As used herein, the term ‘______ ’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. §112, sixth paragraph.

### The Application System

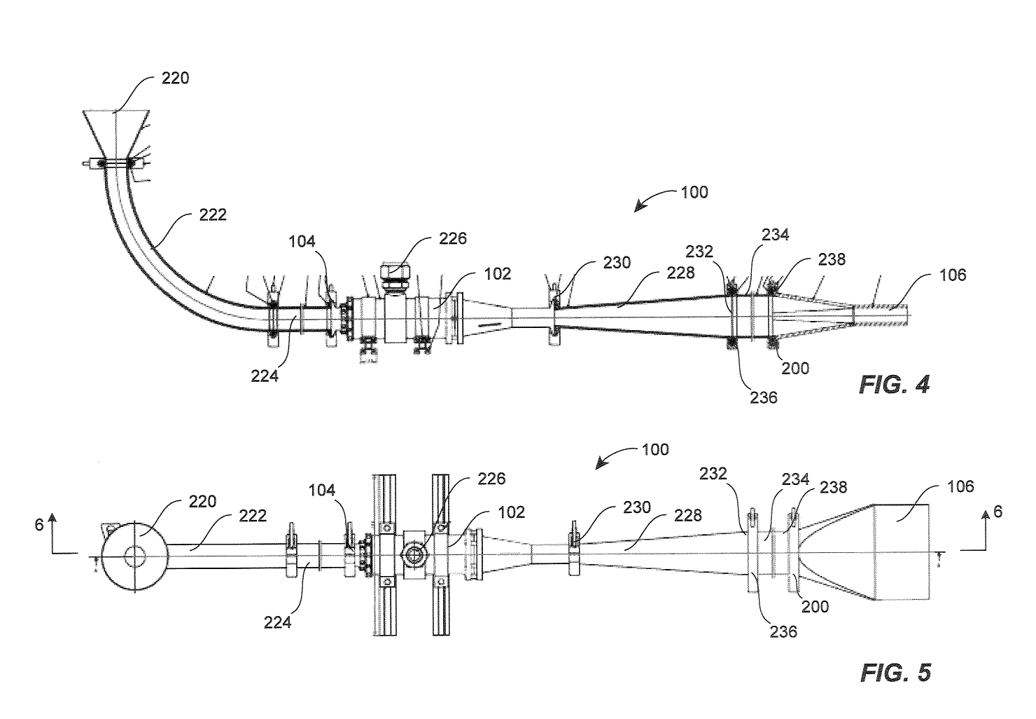

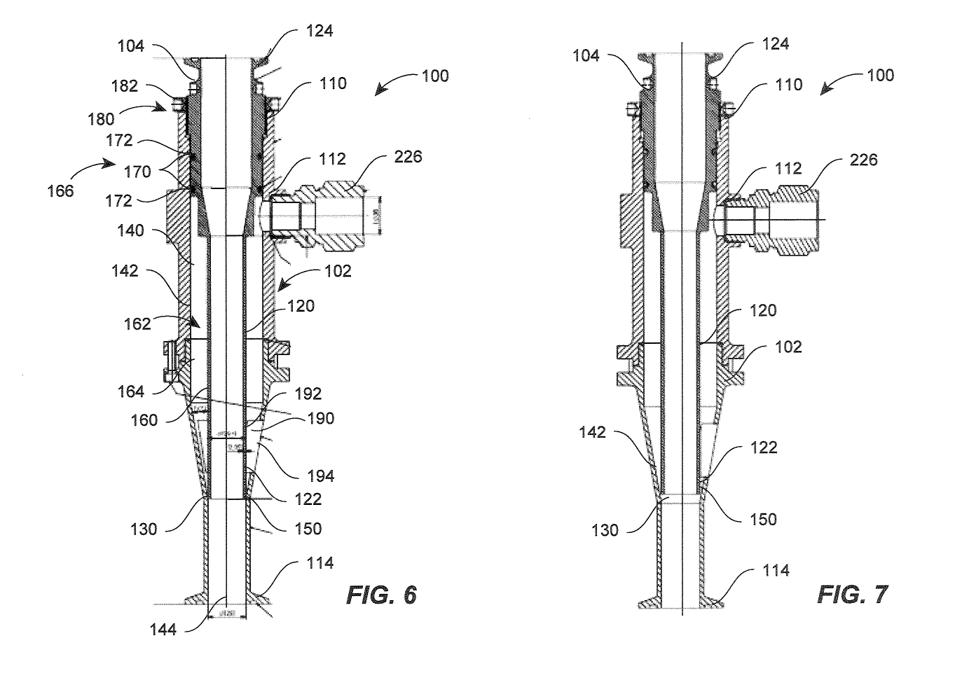

FIGS. 4-10 illustrate a first embodiment of an application system 100 for use with a solid particle material, including superabsorbent polymer (SAP) particles. The application system 100 includes an injector housing 102, an occluder 104 (as better seen in FIGS. 6 and 7), and a nozzle 106. The application system 100 may include other structures as well, as described in greater detail below. While the application system 100 advantageously includes the injector housing 102, occluder 104 and nozzle 106, the system 100 may include only the injector housing 102 and occluder 104 or the nozzle 106. Thus, it will be recognized that these elements could be used separately from each other.

Referring now to FIGS. 6 and 7, the injector housing 102 has a material inlet 110, a gas inlet 112, and a material outlet 114 downstream of the material inlet 110. The occluder 104 is moveably disposed within the injector housing 102 between the gas inlet 112 and the material outlet 114. In particular, the occluder 104 may be in the form of a tube 120 having a first end 122 and a second end 124. As illustrated, the first end 122 may be disposed entirely within the injector housing 102, while the second end 122 depends from the injector housing 102 through the material inlet 110.

The injector housing 102 and the occluder 104 define therebetween at least one adjustable aperture 130 downstream of the material inlet 110 and in fluid communication with the gas inlet 112 and the material outlet 114. The aperture 130 has a first open area with the occluder 104 in a first position relative to the injector housing 102 (see FIG. 6). The aperture 130 may also have a second open area with the occluder 104 in a second position relative to the injector housing 102, the second open area being different than the first open area (see FIG. 7). As illustrated, the first open area may be so small as to be effectively closed, such that the second open area is significantly larger than the first open area.

More particularly, with reference to FIG. 6, the injector housing 102 has a bore 140, which bore 140 is defined by an internal surface 142 of the injector housing 102. The first end 122 of the tube 120 that is disposed within the injector housing 102 is disposed within the bore 140. As illustrated, the bore 140 has a longitudinal axis 144, and the tube 120 is disposed along the longitudinal axis 144. It will be recognized that according to other embodiments the tube 120 may not be aligned with the longitudinal axis 144 of the bore 140.

The tube 120 has a rim 150 disposed about the first end 122. According to the exemplary embodiment illustrated, the adjustable aperture 130 is defined between the internal surface 142 of the injector housing 102 and the rim 150 of the tube 120. It will be recognized that the aperture 130 thus defined has an annular or ring-like shape.

It will also be recognized, however, that the injector housing 102 and the occluder 104 are not limited to only those structures illustrated herein. For example, according to an alternative embodiment, the housing 102 may define one or more tubular passages connected to the gas inlet, each passage having an outlet. According to such an embodiment, the occluder may include one or more plates, which plate or plates may cooperate with the outlets of the passages to define one or more apertures and may be moveable so as to be disposed over the outlets to vary the open space of the apertures so defined. Such an embodiment would also be within the scope of the present disclosure.

Continuing on with reference to FIGS. 6 and 7, the tube 120 also has an external surface 160. The external surface 160 is spaced from the internal surface 142 of the injector housing 102, at least in part over the region 162. The spaced surfaces 142, 160 define a chamber 164 therebetween, which is also annular or ring-like in shape. The chamber 164 is in fluid communication with the gas inlet 112 and the adjustable aperture 130.

The tube 120 also has a second region 166 that is not so spaced from the internal surface 142 of the bore 140, which region is upstream of the first end 122 of the tube 120 and the first region 162. In the second region 166, the surface 160 nearly abuts or abuts the internal surface 142. In this region 166, at least one seal 170 may be disposed between the internal surface 142 of the injector housing 102 and the external surface 160 of the tube 120. As illustrated, two such seals 170 may be disposed between the surfaces 142, 160.

In particular, the tube 120 may have one or more grooves 172 formed in the external surface 160. The seals 170, which may be in the form of an elastomeric O-ring, may be disposed in the grooves 172. In this fashion, the seals 170 may be disposed between the external surface 160 of the tube 120 and the internal surface 142 of the injector housing 102.

The tube 120 also has a third region 180 where, like the second region 166, the exterior surface 160 is not spaced from the surface 142 of the injector housing 102 in the same fashion as the surface 160 is spaced in the first region 162. Unlike the second region 166, however, the surface 160 in the third region 180 cooperates with the surface 142 to attach the tube 120 to the injector housing 102. In particular, the surface 160 is threaded in the region 180, and a mating section 182 of the surface 142 is threaded in a similar fashion. This threaded engagement of the surfaces 142, 166 in the region 180 moveably attaches the tube 120 to the injector housing 102.

A ring 182 is disposed about the second end 124 of the tube 120 in the region 180. The ring 182 has a threaded internal surface that cooperates with the threaded region 180 of the external surface 160 of the tube 120. Movement of the ring 182 about the axis 144 causes the tube 120 to move along and about the axis 144 through the interaction between the threads of the ring 182 and the threaded region 180 of the tube 120.

The tube 120 is thus cantilevered into the bore 140 from its second end 124, with the first end 122 depending into the bore 140. To assist in supporting the first end 122 of the tube 120 and maintaining it centered along the axis 144, one or more supports 190 are disposed in the space 164 between the tube 120 and the injector housing 102. The supports 190 each have a first end 192 attached to the injector housing 102, and a second end 194 proximate to the external surface 160 of the tube 120. As illustrated, the supports 190 may be in the form of triangular-shaped plates; however, the supports 190 are not so limited in all embodiments, and may include other structures as well. Further, while the supports 190 are discussed as being proximate to the external surface 160 of the tube 120, the supports 190 may also abut the external surface 160 at one or more points.

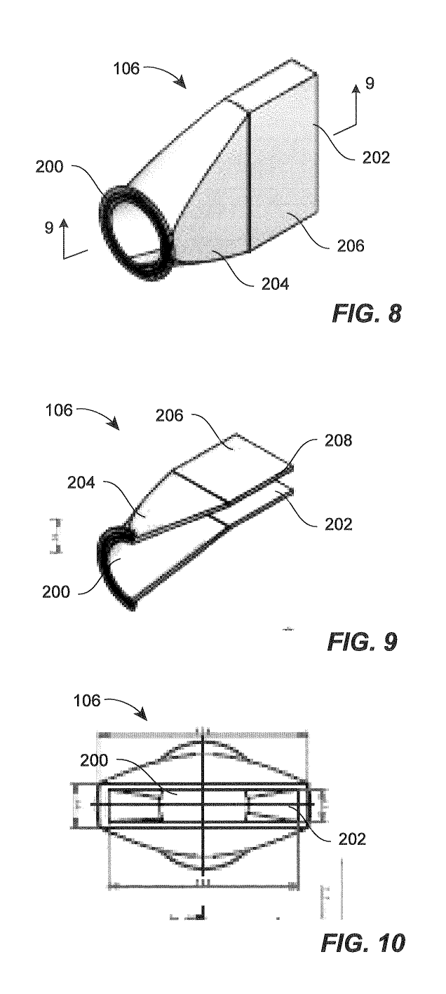

Having thus discussed the structure and operation of the injector housing 102 and the occluder 104, reference is now made to FIGS. 8-10 relative to the structure of the nozzle 106. The nozzle 106 has a nozzle inlet 200, which may be coupled to the material outlet 114 of the injector housing 102, and a nozzle outlet 202. As illustrated, the inlet 200 has a circular cross-section (see FIGS. 8 and 9), while the outlet 202 has a rectangular cross-section (see FIG. 10).

It will be recognized that the nozzle 106 in fact has two sections 204, 206. In the first section 204, the transition is made between the circular cross-section of the inlet 200 to the rectangular cross-section of the outlet 202 through the use of curved surfaces that gradually change the cross-sectional shape between the circle and the rectangle, passing in a continuous fashion through a plurality of intermediate cross-sections of different shapes. In the second section 206, a conduit 208 (see FIG. 9) of rectangular cross-section extends from the first section 204 to the outlet 202.

The outlet 202 has a length (the longer dimension from left to right in FIG. 10) and a width (the shorter dimension from top to bottom in FIG. 10). It will be recognized that the length and width may vary according to the particular product that the application system 100 is used to manufacture. However, according to one exemplary embodiment of the system 100, the length may be between about 80 mm and about 250 mm and the width may be between about 20 mm and about 65 mm. According to another exemplary embodiment, the length may be about 168 mm and the width about 27 mm for adult incontinent products, and the length may be about 90 mm and the width about 49 mm for baby diapers.

Having thus discussed the injector housing 102, occluder 104 and nozzle 106, the remainder of the application system 100 is now discussed with reference to FIGS. 4 and 5.

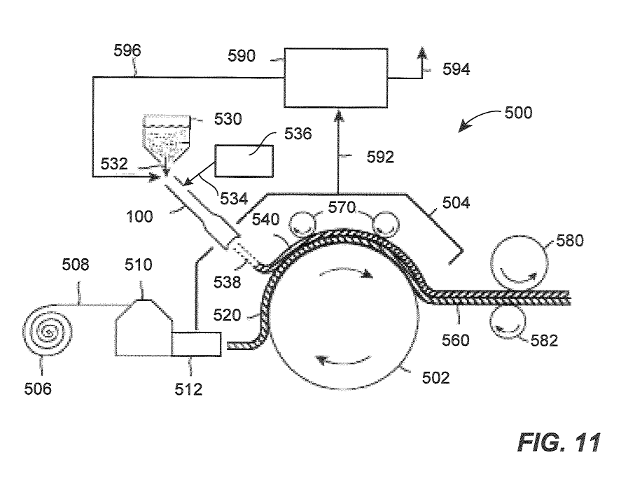

Starting at the left-hand side of the figures, a funnel 220 is attached at one end to a curved conduit 222, which is in turn attached to a straight conduit 224. The straight conduit 224 has been show in broken view, in consideration of the fact that the straight conduit 224 may be significantly longer than the other elements of the application system 100. The funnel 220 may be disposed adjacent to a hopper filed with a volume of solid particle material (see FIG. 11) so as to couple the hopper to the material inlet 110 of the injector housing 102 via the conduits 222, 224. While not shown in this FIGS. 4 and 5, the hopper would also be considered to be part of the application system 100.

Passing along the injector housing 102, it will be noted that a fitting 226 is attached to the injector housing 102. The fitting 226 is in fluid communication with the gas inlet 112 of the injector housing 102 (see FIGS. 6 and 7). The fitting 226 may be connected to a source of pressurized air (see FIG. 11), so as to coupled the source of pressurized air to the gas inlet. Alternatively or in addition, the fitting 226 may be connected to a source of steam. While not shown in this FIGS. 4 and 5, the source of pressurized air would also be considered to be part of the application system 100.

Further down the system 100, a diffuser 228 may be positioned at the material outlet 114. The diffuser 228 may have a diffuser inlet 230 attached to the material outlet 114 of the injector housing 102. The diffuser 228 may also have a diffuser outlet 232 coupled to the nozzle inlet 200. Specifically, a conduit 234 may have a conduit inlet 236 attached to the diffuser outlet 232, and a conduit outlet 238 attached to the nozzle inlet 200. Similar to the conduit 224, the conduit 234 has been show in broken view, in consideration of the fact that the conduit 234 may be significantly longer than the other elements of the application system 100.

### The Solid Particle Material

The solid particle material applied using the application system 100 may include SAP particles, which SAP particles are useful in absorbing liquid material. The particles can have any desired shape such as, for example, cubic, rod-like (e.g., fibers), polyhedral, spherical or semispherical (e.g., granules), rounded or semi-rounded (e.g., droplet-shaped, with or without an internal void), plate-like (e.g., flakes), angular, irregular, and the like. SAP particles generally have particle sizes ranging from about 100 μm to about 850 μm, although particles as small as about 45 μm can also be present. The weight-average particle size for the SAP particles is generally in the range of about 150 μm to about 600 μm. When SAP particles having a non-spherical or non-hemispherical shape are used, the particle sizes are such that the smaller particles in the distribution have a volume equivalent to a sphere of about 100 μm and the larger particles in the distribution have a volume equivalent to a sphere of about 850 μm.

The SAP particles are generally formed from a lightly crosslinked polymer capable of absorbing several times its own weight in water, saline, urine and/or other liquids. SAP particles can be made by conventional processes for preparing SAPs, which processes are well known in the art and include, for example, solution polymerization and inverse suspension polymerization. SAP particles useful with the application system 100 are prepared from one or more monoethylenically unsaturated compounds having at least one acid moiety, such as carboxyl, carboxylic acid anhydride, carboxylic acid salt, sulfonic acid, sulfonic acid salt, sulfuric acid, sulfuric acid salt, phosphoric acid, phosphoric acid salt, phosphonic acid, or phosphonic acid salt. Suitable monomers include acrylic acid, methacrylic acid, maleic acid, fumaric acid, maleic anhydride, and the sodium, potassium, and ammonium salts thereof. Especially preferred monomers include acrylic acid and its sodium salt.

In addition to the SAP particles, the solid particle material may include fluff. Fluff assist in creating an applied layer of solid particle material has an entangled structure with good capillary properties, thereby increasing the absorption efficiency of a product made from the composite of the solid particle material and the substrate. Specifically, the fluff helps transport liquid material (e.g., urine waste in a diaper) via capillary action away from a top surface of a composite into the composite's interior, where the liquid material can be absorbed by the sub-surface SAP particles.

Fluff includes both natural material, such as cellulosic fibers, and synthetic materials, such as polymeric fibers. Cellulosic fibers can include, but are not limited to, chemical wood pulps such as sulfite and sulfate (sometimes called Kraft) pulps, as well as mechanical pulps such as ground wood, thermomechanical pulp and chemithermomechanical pulp. More particularly, the pulp fibers may include cotton, other typical wood pulps, cellulose acetate, debonded chemical wood pulp, and combinations thereof. Pulps derived from both deciduous and coniferous trees can also be used. Additionally, the cellulosic fibers may include such hydrophilic materials as natural plant fibers, milkweed floss, cotton fibers, microcrystalline cellulose, microfibrillated cellulose, polysaccharide fibers (e.g., sugar cane fibers), or any of these materials in combination with wood pulp fibers. Suitable cellulosic fluff fibers include, for example, NB480 (available from Weyerhaeuser Co., Federal Way, Wash.); NB416 (a bleached southern softwood Kraft pulp; available from Weyerhaeuser Co.); CR 54 (a bleached southern softwood Kraft pulp; available from Bowater Inc., Greenville, S.C.); SULPHATATE HJ or RAYFLOC JLD (a chemically modified hardwood pulp; available from Rayonier Inc., Jessup, Ga.); NF 405 (a chemically treated bleached southern softwood Kraft pulp; available from Weyerhaeuser Co.); and CR 1654 (a mixed bleached southern softwood and hardwood Kraft pulp; available from Bowater Inc.). Suitable polymeric fibers include polyolefins (e.g., polypropylenes), rayons, and polyesters, and are available from Freudenberg Nonwovens (Charlotte, N.C.), PGI Nonwovens (Charlotte, N.C.), and Rayonier, Inc. (Jessup, Ga.).

The SAP particles, fluff or other fiber-like materials are included in an amount such that the basis weight of the SAP particles and fluff combined is generally in a range of about 400 g/m2 to about 1200 g/m2. The SAP particles are generally included in a composite in a range of about 5 wt. % to about 80 wt. %, for example about 25 wt. % to about 55 wt. %, relative to the combined weight of the SAP particles and fluff included in the composite. Similarly, the fluff is generally included in the composite in a range of about 20 wt. % to about 95 wt. %, for example about 45 wt. % to about 75 wt. %, relative to the combined weight of the SAP particles and fluff included in the composite.

The solid particle material may also include a binder. Any included binder can attach to the outer surfaces of the SAP particles, facilitating the attachment of the SAP particles to each other and to the fluff. The binder can be in the form of solid binder particles generally having particle sizes ranging from about 10 μm to about 30 μm, for example from about 15 μm to about 25 μm. Suitable binders include natural organic binders (for example, starch and other polysaccharides), water-based adhesives, and hot-melt adhesives. A suitable polysaccharide-based binder is available from Lysac Technologies, Inc. (Boucherville, Canada).

When included, the solid binder is generally added at a flow rate of about 0.005% to about 40% of the flow rate of SAP particles. The flow rate of binder can be selected independently from the flow rates of the SAP particles. The particular amount of binder used is selected such that each of the SAP particles issuing application system 100 ideally has at least some binder coated to its outer surface prior to being deposited on the substrate. In practice, however, up to about 20% by number (for example, up to about 10%) of the SAP particles can be free of binder. Binder-free SAP particles can still be successfully deposited onto the substrate, due to the likelihood of being deposited adjacent to SAP particles that have been successfully coated with the binder. For those SAP particles that are coated with binder, about 5% to about 80% (for example about 30%) of the surface area of each individual SAP particle is coated.

The fluff material, because of its self-entangling fibrous structure, need not be coated with binder to form an at least loosely coherent structure. Thus, a binder flow rate that results in the desired degree of coverage for the SAP particles (i.e., with respect the number fraction of SAP particles that are coated and the surface area fraction of each SAP particle that is coated with binder) is sufficient to result in the components of an applied particle layer being suitably adhered to each other in the particle-substrate composite.

### The Application System in Use

The application system 100 can be used in a process for the application of the SAP particles to a substrate. An exemplary production system 500 is illustrated in FIG. 11, with the application system 100 integrated therewith. The production system 500 includes a rotating vacuum forming drum 502 partially encased by a forming chamber 504. In an alternate embodiment (not shown), the forming drum 502 can be replaced by a horizontal endless belt.

A virgin fluff roll 506 feeds a continuous sheet of virgin fluff 508 to a hammer mill 510. The virgin fluff 508 can be formed from the same materials described above for the fluff material that is optionally fed to the application system 100. However, the virgin fluff 508 and the optional fluff in the application system 100 need not be formed from the same materials in a single application. The virgin fluff 508 is preferably formed from polymeric fibers. The continuous sheet of virgin fluff 508 is fiberized into shorter, discontinuous fibers by the hammer mill 510. The fiberized virgin fluff is then fed via a hammer mill applicator 512 into the forming chamber 504.

The fiberized virgin fluff entering the forming chamber 504 is applied to the outer surface of the rotating vacuum forming drum 502. The rotation and vacuum of the forming drum 502 results in a continuous layer of fiberized virgin fluff on the outer surface of the forming drum, thereby forming a substrate 520 and further conveying the substrate 520 through the forming chamber 504.

The application system 100 is situated such that the nozzle outlet 202 is located in the forming chamber 504 and directed toward the forming drum 502. The application system 100 may include, as noted above, a feed hopper 530 containing a volume of solid particle material. A metering device (such as a screw feeder, for example as manufactured and sold by Acrison, Inc. of Moonachie, N.J.) delivers the desired amount of solid particle material in a solids feed stream 532 to the funnel 220. A gas stream 534 is delivered to the gas inlet 112 of the injector housing 102 via the fitting 226. Where the gas used is air, the stream 534 may be provided by a source 536 of pressurized air that is coupled to the fitting 226, as also mentioned above. If optional components (e.g., fluff, binders) are delivered by the application system 100, additional feeding means (not shown) may be included in the process. The solid particle material enters the forming chamber 504 at 538 and is then deposited as a particle layer 540 on the substrate 520, thereby forming the particle-substrate composite 560.

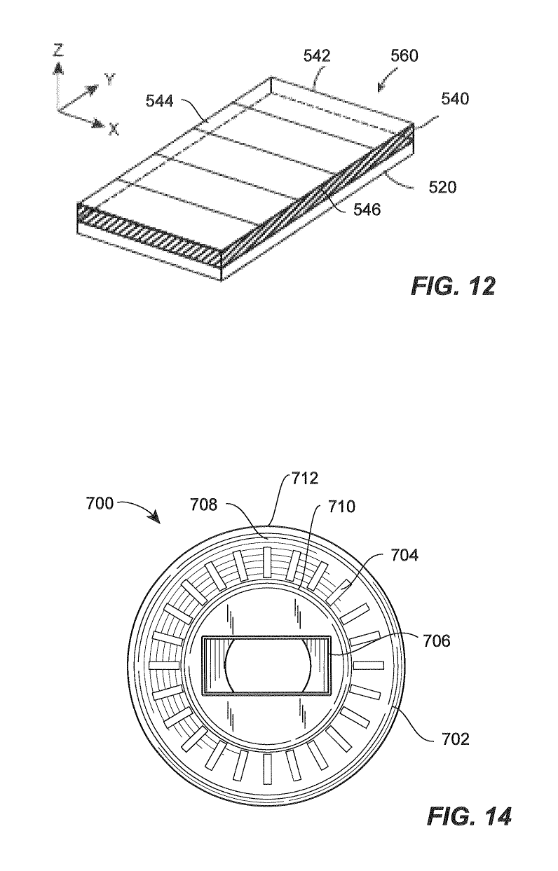

It is believed that the application system 100 will provide a substrate similar to that illustrated in FIG. 12. That is, the particle layer 540 is applied to the substrate 520 such that the height 542 of the particle layer 540 is substantially constant over time, leading to a uniform application height to the substrate 520 which is advancing the y-direction in FIG. 12. This is to be compared with the profile of the layer produced using conventional methods and apparatuses, as illustrated in FIG. 3, wherein the application height (in the z-direction) may fluctuate significantly over time the feed direction (y-direction).

It should also be noted that the layer in FIG. 3 varies between the ends of the particle layer (in the x-direction). This is another way in which the particle layer 540 applied using the application system 100 differs from that generated using conventional methods and apparatuses: the layer 540 maintains a uniform height between ends 544, 546. In fact, the layer 540 applied using the application system 100 may have a significant and distinguishable termination of the layer 540 at the ends 544, 546. This should be contrasted with the layer produced using conventional methods and apparatuses, wherein the layer has poorly defined edges. This is particularly advantageous where only a particular section of the substrate 520 is to be covered with the particle layer 540, reducing rejections for application outside the defined target region.

As the particle-substrate composite 560 is conveyed through the forming chamber 504 by the forming drum 502, scarfing rolls 570 optionally can be used to remove and recycle excess material from the particle layer 540. The scarfing rolls 570 can improve the weight distribution deviation of the composite 560 by removing material from the particle layer 540 in regions of the composite 560 having locally high deposition amounts. However, the scarfing rolls 570 are ineffective for improving the weight distribution deviation in regions of the composite 560 having locally low deposition amounts (i.e., below the level of the scarfing rolls 570). The application system 100 is capable of applying the solid particle material to the substrate 520 in a manner that reduces the weight distribution deviation of the composite 560 without using the scarfing rolls 570. Accordingly, the scarfing rolls 570 can be omitted from the production process.

When the particle-substrate composite 560 exits the forming chamber 504, it is removed from the forming drum 504 via a vacuum transfer drum 580. The composite 560 is then conveyed downstream via transfer drums 580, 582 for further processing steps (not shown), such as cutting, application of other absorbent article components (e.g., films, adhesives, elastics, nonwovens), and packaging of a final absorbent article product (e.g., diaper or a feminine hygiene product).

In the illustrated embodiment of FIG. 11, a vacuum is drawn within the forming chamber 504 via a rotary dust collecting system 590. The vacuum creates a total airflow of about 7000 standard cubic feet per minute (scfm) to about 16000 scfm cycling through the forming chamber 504. A forming chamber exhaust 592 removes dust and other solids (including, e.g., fiberized virgin fluff, SAP particles, optional fluff and/or binder delivered by the application system 100) that is airborne in the headspace of the forming chamber 504 and delivers the dust and other solids to the rotary dust collecting system 590. The rotary dust collecting system 590 uses rotary filters (not shown) to expel waste (e.g., dust) from the process via a process exhaust 494. Non-waste (e.g., fiberized virgin fluff, SAP particles, optional fluff and/or binder) is recycled by the rotary dust collecting system 590 via a process recycle 596. In an embodiment (not shown), the process recycle 596 can be fed directly into the forming chamber 504. However, in the illustrated embodiment, the process recycle 596 is combined with the solids feed stream 532 and the two are then delivered by the application system 100 to the forming chamber 504. This combination of streams has the advantage of providing an increased flow residence time over which the recycled and fresh feed material are pre-blended prior to entering the forming chamber, thereby increasing the homogeneity of the final particle-substrate composite 560.

One advantage peculiar to the applicator system 100, as illustrated, is the ability of the system 100 to be configured to provide different flow rates and different pressures through the adjustability of the aperture 130. In particular, it will be recognized that a process for using the system 100 to provide this adjustability may include disposing the occluder 104 in a first position relative to the injector housing 102 so that the aperture 130 has a first open area, passing air through the aperture 130, drawing solid particle material into the injector housing 102 at a first rate, and ejecting the solid particle material from the nozzle outlet 202 onto a substrate. Further, the process may include moving the occluder 104 to a second position relative to the injector housing 102 so that the aperture 130 has a second open area, the second open area being different than the first open area. The process may also include passing air through the aperture 130, drawing solid particle material into the injector housing 102 at a second rate, the second rate being different than the first rate, and ejecting the solid particle material from the nozzle outlet 202 onto a substrate.

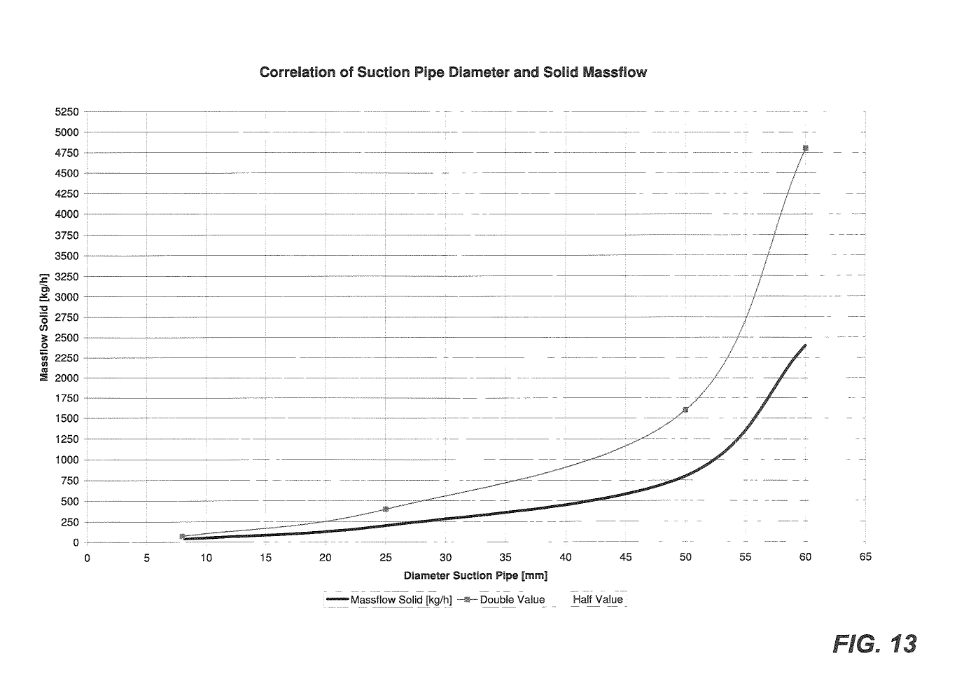

FIG. 13 illustrates a correlation between the diameter of the tube 120 and the mass flow rate of the solid particle material (the rate at which material is drawn into the injector and ejected from the nozzle outlet) in such a process. According to exemplary embodiments of the present disclosure, the superabsorbent polymer (SAP) mass flow rate may be between about 80 kg/hour and about 2000 kg/hour. However, the SAP mass flow rate may also be between about 180 kg/hour and about 600 kg/hour, or even more particularly between about 210 kg/hour and about 480 kg/hour. However, the operation of the process below or above the area illustrated in FIG. 13 may still be possible, even though undesirable effects may occur, such as very high pressure loss and/or segregation of gas and particles.

### An Application System Variant

As mentioned previously, the embodiment illustrated in FIGS. 4-10 is merely exemplary, and may be modified while remaining within the teaching of the present disclosure even though the modification may not be particularly illustrated herein. However, one variant of the application system 100 is illustrated in FIG. 14. According to this variant, the application system 100 is combined with a two-component system, such as is disclosed in PCT Publication No. WO 2008/068220, which claims priority to U.S. Provisional Application No. 60/872,942 and designates the United States, and which is incorporated in its entirety for all purposes herein.

As shown, the variant system 700 includes an adapter 702 that may include a structure similar to the nozzle 106 illustrated in FIGS. 8-10. The adapter 702 defines two separate systems through which material streams may flow. A first material stream exits the adapter 702 through apertures 704, while a second material stream exits through a rectangularly-shaped nozzle outlet 706 disposed at the center of the adapter 702. Because the apertures 704 are disposed further from a longitudinal axis of the adapter 702 than the nozzle outlet 706, the material stream exiting the apertures 704 may be referred to as the outer stream, while the material stream exiting the nozzle outlet 706 may be referred to as the inner stream. The inner stream would be the stream exiting the system 100 described above, for example.

The apertures 704 are formed in a plate 708 having an inner edge 710 and an outer edge 712. The plate 708 is angled between the inner edge 710 and the outer edge 712 relative to a longitudinal axis of the adapter 702. Specifically, the plate 708 is angled to generate converging streams: the outer stream exiting the apertures 704 is directed toward the inner stream exiting the nozzle outlet 706 so as to mix with the inner stream in a free stream region downstream from the adapter 702. The mixing of the streams in a converging fashion is believed both to improve the uniformity of the solid particles applied by the system 700 and to improve the mixing of the outer stream with the inner stream.

The outer stream may include, for example, water and/or steam. The inclusion of water may reduce the accumulation of electrostatic charges on the solid particles and the fluff. The water may further facilitate the attachment of binders to the solid particles. Moreover, the outer stream may include liquid binders that may not be included in the inner stream.

We claim:

1. An application system for applying a solid particle, the system comprising
an injector housing having a material inlet, a gas inlet, and a material outlet downstream of the material inlet;
an occluder moveably disposed within the injector housing between the gas inlet and the material outlet, the injector housing and the occluder defining therebetween at least one adjustable aperture downstream of the material inlet and in fluid communication with the gas inlet and the material outlet, the aperture having a first open area with the occluder in a first position relative to the injector housing and the aperture having a second open area with the occluder in a second position relative to the injector housing, the second open area being different than the first open area; and
a nozzle having a nozzle inlet coupled to the material outlet of the injector housing and a nozzle outlet, the inlet having a circular cross-section and the outlet having a rectangular cross-section.

2. The application system according to claim 1, wherein the solid particle comprises superabsorbent polymer (SAP) material.

3. The application system according to claim 1, wherein:
the injector housing comprises a bore with a longitudinal axis; and the occluder comprises a tube that has at least a first end disposed within the bore and that moves axially along the longitudinal axis between the first and second positions.

4. The application system according to claim 3, wherein the injector housing has an internal surface that defines the bore and the tube has a rim disposed about the first end, the adjustable aperture defined between the internal surface of the injector housing and the rim of the tube.

5. The application system according to claim 4, wherein the tube has an external surface spaced from the internal surface of the injector housing, at least in part, to define a chamber therebetween, the chamber in fluid communication with the gas inlet and the adjustable aperture.

6. The application system according to claim 5, further comprising at least one seal disposed between the internal surface of the injector housing wall and an external surface of the tube upstream of the first end of the tube.

7. The application system according to claim 5, further comprising supports with a first end attached to the injector housing and a second end proximate to the external surface of the tube to center the tube within the bore.

8. The application system according to claim 3, wherein the tube is connected to the injector housing by a threaded connection so that the tube moves along and about the longitudinal axis.

9. The application system according to claim 1, further comprising a source of pressurized air coupled to the gas inlet.

10. The application system according to claim 9, further comprising a hopper coupled to the material inlet, and a volume of solid particle material disposed in the hopper.

11. The application system according to claim 1, further comprising a diffuser with a diffuser inlet attached to the material outlet of the injector housing and a diffuser outlet coupled to the nozzle inlet.

12. The application system according to claim 11, further comprising a conduit with a conduit inlet attached to the diffuser outlet and a conduit outlet attached to the nozzle inlet.

13. The application system according to claim 1, wherein the nozzle outlet has a length of between 80 mm and 250 mm and a width of between 20 mm and 65 mm.

14. A process for applying a solid particle material to a substrate using an application system including a injector housing having a material inlet, a gas inlet, and a material outlet downstream of the material inlet, an occluder moveably disposed within the injector housing between the gas inlet and the material outlet, the injector housing and the occluder defining therebetween at least one adjustable aperture downstream of the material inlet and in fluid communication with the gas inlet and the material outlet, and a nozzle having a nozzle inlet coupled to the material outlet of the injector housing and a nozzle outlet, the inlet having a circular cross-section and the outlet having a rectangular cross-section, the process comprising:
disposing the occluder in a first position relative to the injector housing so that the aperture has a first open area;
passing air through the aperture;
drawing the solid particle material into the injector housing at a first rate;
ejecting the solid particle material from the nozzle outlet onto a substrate;
moving the occluder to a second position relative to the injector housing so that the aperture has a second open area, the second open area being different than the first open area;
passing air through the aperture;
drawing the solid particle material into the injector housing at a second rate, the second rate being different than the first rate; and
ejecting the solid particle material from the nozzle outlet onto a substrate.

15. The process for applying a solid particle material according to claim 14, wherein the solid particle material is a superabsorbent polymer (SAP) material.

16. The process for applying a solid particle material according to claim 14, wherein the mass flow rate through the system is between about 80 kg/hour and about 2000 kg/hour.

17. The process for applying a solid particle material according to claim 16, wherein the mass flow rate through the system is between 210 kg/hour and 480 kg/hour.

18. An absorbent article formed according to the process of claim 14.
